(19) United States
(12) Patent Application Publication
(10) Pub. No.: US 2004/0028731 A1
(43) Pub. Date: Feb. 12, 2004

(54) Delivery mechanism

(76) Inventor: Rex Munday, Hamilton (NZ)

Correspondence Address: NIXON PEABODY, LLP, 401 9TH STREET, NW, SUITE 900, WASINGTON, DC, 20004-2128, US

(21) Appl. No.: 10/276,250

(22) PCT Filed: May 9, 2001
(86) PCT No.: PCT/NZ01/00081

(30) Foreign Application Priority Data
May 18, 2000 (NZ) 504631

(51) Int. Cl. B29C 47/00; A61K 9/22

(52) U.S. Cl. 424/468; 264/148

(57) ABSTRACT

The present invention relates to a delivery mechanism for the introduction of a biological agent to an animal characterised in that the delivery mechanism is a bolus which contains the biological agent. In preferred embodiments of the present invention the biological agent is Duddingtonia flagrans and the animal to which the mechanism is introduced is a sheep.

### TECHNICAL FIELD

This invention relates to a delivery mechanism.

Reference throughout this specification shall be made to the use of the present invention as a delivery mechanism for the introduction of biological substances to animals.

Reference throughout this specification shall be made to the biological substance as being a fungus such as Duddingtonia flagrans. It should be appreciated however that the principles of the present invention could apply to other biological substances as well.

### BACKGROUND ART

Considerable research has been conducted into the use of biological agents such as fungi to treat animals. One such agent is the fungus Duddingtonia flagrans which can be used to reduce the population of nematode parasites on pasture. PCT Patent Application No. PCT/DK92/00269 (Wolstrup et al) discusses this in detail.

Reduction of parasites is achieved by introducing the fungus to an animal such that the fungus passes through the animal and is excreted in the animal's faeces. The fungus then grows in the faeces killing off the nematode parasites contained therein. This assists in preventing re-infection of the animals by reducing significantly the population of the parasite in pastures. To ensure that this reduction in parasite numbers is maximised it is desirable for the biological agent to be dosed into the animal regularly and over an extended time period, ideally 4-10 weeks.

Unfortunately, a number of problems arise with current methods used to introduce biological agents to animals.

Biological agents are intrinsically more labile particularly with respect to their environment than inert, synthetic or mineral substances and therefore it is much more difficult to find a delivery mechanism which is labour and cost effective while still maintaining the viability of the agent.

Biological agents have been added to the water, feed, feed supplements and mineral supplements of animals. However, this is a haphazard method of introducing these agents, as the amount of feed, feed supplement and water ingested by an animal is variable. Also, the feed and water supply which contains the agent is usually not the sole source of food or water. Therefore, the actual dosage of biological agent that each animal receives is highly variable which can cause variations in the degree of effect recorded with the biological agent.

Another problem with introducing the agent in water or other similar substrates/media is that the agent may be prematurely activated. For example, if the agent is in the form of a spore, water may cause the spore to germinate before ingestion by the animal. If the aqueous environment does not support the growth of the organism, it may die before ingestion by the animal or in the animal before the organism is delivered to where it is intended to function. Such germination may also cause problems within the animal as some organisms are known to produce toxic metabolites during their growth stages which may cause health issues if ingested or produced in the animal.

If the function of the biological agent is to act in relation to the animal's digestive system, then the agent cannot be introduced by a number of other methods used in the treatment of animals. For example, methods which would be unsuitable include external application of therapeutic, injection and so forth.

Another problem with the introduction of biological agents is that the delivery mechanism itself must provide an environment that is not toxic to the agent.

Another problem with conventional delivery mechanisms is that they are short term in nature, being processed rapidly by the animal. Ideally, biological agents need to be introduced to the animal at a controlled dose over a period of time in a manner which is not labour intensive.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a delivery mechanism for the introduction of a biological agent to an animal characterised in that the delivery mechanism is a slow release device which contains the biological agent.

In preferred embodiments of the present invention, the delivery mechanism is one that can be used to deliver the biological agent to the digestive system of the animal. However, it should be appreciated that it is possible the present invention could be used to deliver agents to other sites, for example intravaginally.

The biological agent may be any living organism, or entity capable of life, and may include such agents as micro-organisms, eggs, worms, insects or any life stages thereof which may be appropriate and so forth. It is envisaged however that in most embodiments the biological agent is one that is beneficial to the animals directly or indirectly.

In preferred embodiments the biological agent is a micro-organism. Preferably it is a bacterium, protozoan or fungus. In particular it is a fungus, especially the fungus Duddingtonia flagrans. Reference throughout this specification shall now be made of use of the present invention in relation to Duddingtonia flagrans. This fungus has important properties for which this invention is highly suited. It should however be appreciated that the present invention can also be applied to other fungi or biological agents.

Reference throughout his specification shall now be made to the animal to which the agent is delivered as being a sheep. Again, it should be appreciated that the present invention can be delivered to many different animal species including for example ruminant domestic animals such as cattle, goats and so forth.

In preferred embodiments of the present invention the slow release device is a bolus.

The term bolus is one which is well understood in the animal remedy trade. While a dictionary meaning of bolus is a large pill or tablet, commonly a bolus is in the form of an elongate cylinder designed to slowly dissolve in the rumen of the animal. Boluses are generally delivered into the rumen by use of a bolus applicator which delivers the bolus to the top of the animal's oesophagus, after which it is swallowed by the animal.

The use of a bolus as a delivery mechanism for biological agents solves a number of the problems associated with the prior art.

One advantage is that by having a discrete mechanism with a known release profile, the amount of biological agent which is delivered can be accurately known. This makes the treatment and the analysis of the effects of treatment of the animal much more precise than previously.

A bolus is comprised of a solid matrix generally coated in an impervious material having an opening through which the active material can be released. Therefore it is unlikely that there will be premature activation of the biological agent until the bolus is within the animal and the agent released and thereby exposed to the rumen liquor. This saves wastage and again gives greater accuracy in the treatment of the animal.

Boluses in general are designed to release their contents gradually over a period of time. The use of this mechanism therefore saves a considerable amount of labour and expense by allowing biological agents to be delivered in one application, but to act over a period of time. For example, most agricultural practices have large numbers of animals requiring treatment. Traditional delivery mechanisms such as drenches and injections require frequent applications as their effect may be short lived. It can be seen that frequent applications multiplied over a large number of animals results in a significant amount of labour, time and expense. In contrast, the present invention enables a single application per animal to last for a significant period of time. It therefore should be apparent that the savings in labour and expense will be considerable.

There are problems however with the introduction of biological agents into boluses due to the delicate nature of the agent and the requirement that the viability of the agent is preserved. One way of making boluses is by compression and another is by extrusion.

The applicant's patent specification of New Zealand Patent No.278977 refer to a number of methods by which a bolus can be prepared including compression and extrusion—all of which involve heat.

As generally heat is destructive to the viability of many biological agents including fungi such as Duddingtonia flagrans, it is not an obvious decision to introduce a biological agent into a bolus. Suprisingly, the inventor has found, however, that the incorporation of Duddingtonia flagrans into a matrix such as that given in examples later in this text unexpectedly imparts a thermal stability to Duddingtonia flagrans enabling the fungus spores to survive for prolonged periods at elevated temperatures.

One method of making boluses mug the compression technique is discussed below.

First the bolus constituents are melted and mixed together. After cooling, the solidified mass is ground to fine particles which are then compressed to make the bolus.

As can be appreciated, considerable pressures are required to form a bolus using this compression technique. The inventor has found that while some viability of the spores is maintained, this is not a preferred method of manufacture.

The preferred method of manufacture which ensures greater viability of the spores is that of extrusion. This method can involve the following technique described in PCT Application No. PCT/NZ95/00110:

a) mixing the ingredients at a temperature below the melting point of any of the ingredients, and

b) feeding the coarsely mixed ingredients to an extruder which has a number of zones along its axial length, the temperature of each zone being maintained substantially independently, and

c) ensuring that the first zone to which the ingredients are transported is at a temperature below the melting point of any of the ingredients, and

d) ensuring that a subsequent zone is at a temperature higher than the melting point of some of the ingredients, and

e) ensuring that some recirculation of the mixture takes place within the machine, in order to achieve thorough mixing and a high degree of homogeneity within the mix, and

f) allowing the resulting paste to pass through a long passage or die which has walls maintained at a temperature a little above the freezing point of the paste, and

g) ensuring that the passage is long enough that the mixture reaches a high viscosity over the most of the cross-section of the passage, and

h) allowing the resulting paste to pass through a second passage or die which has walls maintained at a temperature a little below the freezing point of the paste, and

i) supplying sufficient pressure that the adhesion between solidifying paste and the die walls is broken and that the mixture is extruded out of the die with a substantially uniform velocity over the whole cross-section of the die, and

j) collecting the extruded rod at the exit of the die, and

k) optionally shaping the end of the rod, and

l) perhaps coating the rod, and

m) cutting the rod to length, and

n) collecting the cut rods for further processing or packing.

A common bolus sold by the applicant having the general composition outlined in NZ Patent No. 278977 is a zinc bolus. However, such a high concentration of zinc is required only in special circumstances, such as in prophylaxis of the disease facial eczema. Therefore, the applicant needed to find an alternative compound to zinc oxide that could be included in the bolus matrix.

This alternate material would have to be inert and non-toxic to the biological agent. It also would have to have a relatively high density in order to encourage the bolus to remain within the rumen.

The substitute substance would have to be relatively cheap, have suitable physical characteristics, be non toxic, and also easily handled in the manufacturing process.

One substance which meets all of the above requirements is barium sulphate.

Preferably a generic composition of the bolus is as follows.

i) a core comprising a substantially homogeneous mixture of:

a) a water insoluble physiologically acceptable binder comprising wax, fat, oil, fatty acid, fatty acid ester, fatty acid aide, fatty acid alcohol or the like organic compound having a melting point above 50° C.;

b) a physiologically acceptable solubilising agent;

c) a biological agent; and

d) where required, a physiologically acceptable inert densifier of sufficient density and in sufficient quantities to give the bolus a minimum density of 1.5 g/cm³; and

ii) a coating of a physiologically acceptable material over substantially all of the surface of the core but leaving exposed a core portion whereby in use liquid in the rumen will dissolve said core allowing release of the beneficial agent into the rumen.

Preferably the binder includes a fatty acid ester which in some instances may be glycerol monostearate.

Preferably the solubilising agent is polyethylene glycol stearate.

Alternatively, the said solubilising agent is a sodium salt of a long chain fatty acid.

Preferably the biological agent is Duddingtonia flagrans.

Some embodiments may include one or more beneficial agents. The beneficial agents may be nutrients, growth promotants or a therapeutic substance, for example an anthelmintic.

Preferably the densifier is iron powder, barium sulphate or iron oxide.

Preferably the bolus is in the shape of a cylinder which is closed at one end and open at the other.

Preferably said closed end is hemispherical in shape.

Specific examples of boluses which work particularly well in accordance with the present invention are given below.

### EXAMPLE 1

A bolus made from a mixture containing between

1 to 20% fungal spores or other biological materials,

68 to 84% barium sulphate, and

10 to 20% physiologically acceptable binding and releasing agents.

### EXAMPLE 2

A bolus made from a mixture containing 60-80% barium sulphate,

5-20% Mono-Di HV40 a trade name for glycerol monostearate non-self-emulsifying, a product of Danisco Ingredients (Malaysia), Penang Malaysia, and

0.5-10% Lipomulse 165 a trade name for glycerol monostearate self-emulsifying, a blend of glycerol monostearate and polyethylene glycol monostearate (a product of Lipo Chemicals Inc., of Paterson, N.J., USA).

A mixture containing between 60 and 99% of the above powder and between 1 and 40% Duddingtonia flagrans spores was pressed at between 2000 and 8000 psi in a suitable die.

Alternatively in preferred embodiments this mixture could be melted and extruded.

### EXAMPLE 3

A more specific example of a bolus mixture which works particularly well is given below.

10% Duddingtonia spores

76.5% barium sulphate

11.7% Mono-Di HV 40 a trade name for glycerol monostearate non-self-emulsifying, a product of Danisco Ingredients (Malaysia), Penang, Malaysia

1.8% glycerol stearate, self-emulsifying Lipomulse 165, a product of Lipo-Chemicals Inc, Paterson, N.J., USA.

It should be appreciated that while these are preferred examples which work particularly well in meeting the criteria discussed previously, other embodiments of the present invention are also envisaged.

Aspects of the present invention have been described by way of example only and it should be appreciated that modifications and additions may be made thereto without departing from the scope of the appended claims.

1. A delivery mechanism for the introduction of a biological agent to an animal over an extended period
characterised in that
the delivery mechanism is a slow release device which contains the biological agent and includes a core comprising a substantially homogeneous mixture of:
a) a water insoluble physiologically acceptable binder comprising wax, fat, oil, fatty acid, fatty acid ester, fatty acid amide, fatty acid alcohol or the like organic compound having a melting point above 50° C.;
b) a physiologically acceptable solubilising agent;
c) a biological agent; and
d) where required, a physiologically acceptable inert densifier of sufficient density and in sufficient quantities to give the bolus a minimum density of 1.5 g/cm³.

2. A delivery mechanism as claimed in claim 1 which includes a coating of a physiologically acceptable material over substantially all of the surface of the core but leaving exposed a core portion whereby in use liquid in the rumen will dissolve said core allowing release of the beneficial agent into the rumen.

3. A delivery mechanism as claimed in either claim 1 or claim 2 which includes the following:
1 to 20% biological agents,
68 to 84% barium sulphate,
10 to 20% physiologically acceptable binding and releasing agents.

4. A delivery mechanism as claimed in any one of claims 1 to 3 wherein the slow release device is a bolus.

5. A delivery mechanism as claimed in any one of claims 1 to 4 wherein the delivery mechanism delivers the biological agent to the digestive system of the animal.

6. A delivery mechanism as claimed in any one of claims 1 to 5 wherein the biological agent is a micro-organism.

7. A delivery mechanism as claimed in any one of claims 1 to 6 wherein the biological agent is a fungus.

8. A delivery mechanism as claimed in claim 7 wherein the biological agent is Duddingtonia flagrans.

9. A delivery mechanism as claimed in any one of claims 1 to 8 wherein the animal is a ruminant.

10. A delivery mechanism as claimed in any one of claims 1 to 9 wherein the animal is a sheep.

11. A delivery mechanism as claimed in any one of claims 1 to 9 wherein the animal is cattle.

12. A delivery mechanism as claimed in any one of claims 1 to 11 which is made by the process of extrusion.

13. A delivery mechanism as claimed in any one of claims 1 to 12 made to the following method:
a) mixing the ingredients at a temperate below the melting point of any of the ingredients, and
b) feeding the coarsely mixed ingredients to an extruder which has a number of zones along its axial length, the temperature of each zone being maintained substantially independently, and
c) ensuring that the first zone to which the ingredients are transported is at a temperature below the melting point of any of the ingredients, and
d) ensuring that a subsequent zone is at a temperature higher than the melting point of some of the ingredients, and
e) ensuring that some recirculation of the mixture takes place within the machine, in order to achieve thorough mixing and a high degree of homogeneity within the mix, and
f) allowing the resulting paste to pass through a long passage or die which has walls maintained at a temperature a little above the freezing point of the paste, and
g) ensuring that the passage is long enough that the mixture reaches a high viscosity over the most of the cross-section of the passage, and
h) allowing the resulting paste to pass through a second passage or die which has walls maintained at a temperature a little below the freezing point of the paste, and
i) supplying sufficient pressure that the adhesion between solidifying paste and the die walls is broken and that the mixture is extruded out of the die with a substantially uniform velocity over the whole cross-section of the die, and
j) collecting the extruded rod at the exit of the die, and
k) optionally shaping the end of the rod, and
l) perhaps coating the rod, and
m) cutting the rod to length, and
n) collecting the cut rods for further processing or packing.

14. A method of manufacturing a delivery mechanism as claimed in any one of claims 1 to 13 characterised by the steps of:
a) mixing the ingredients at a temperature below the melting point of any of the ingredients, and
b) feeding the coarsely mixed ingredients to an extruder which has a number of zones along its axial length, the temperature of each zone being maintained substantially independently, and
c) ensuring that the first zone to which the ingredients are transported is at a temperature below the melting point of any of the ingredients, and
d) ensuring that a subsequent zone is at a temperature higher than the melting point of some of the ingredients, and
e) ensuring that some recirculation of the mixture takes place within the machine, in order to achieve thorough mixing and a high degree of homogeneity within the mix, and
f) allowing the resulting paste to pass though a long passage or die which has walls maintained at a temperature a little above the freezing point of the paste, and
g) ensuring that the passage is long enough that the mixture reaches a high viscosity over the most of the cross-section of the passage, and
h) allowing the resulting paste to pass through a second passage or die which has walls maintained at a temperature a little below the freezing point of the paste, and
i) supplying sufficient pressure that the adhesion between solidifying paste and the die walls is broken and that the mixture is extruded out of the die with a substantially uniform velocity over the whole cross-section of the die, and
j) collecting the extruded rod at the exit of the die, and
k) optionally shaping the end of the rod, and
l) perhaps coating the rod, and
m) cutting the rod to length, and
n) collecting the cut rods for further processing or packing.

15. A delivery mechanism substantially as herein described with reference to and as illustrated by the accompanying examples.

16. A method of manufacturing substantially as herein described with reference to and as illustrated by the accompanying examples.
